(19) United States
(12) Patent Application Publication
(10) Pub. No.: US 2005/0271564 A1
(43) Pub. Date: Dec. 8, 2005

(54) Plasma sterilizer having dehumidifier

(76) Inventor: Jung-Suek Ko, Seoul (KR)

Correspondence Address: Richard L Byrne, 700 Koppers Building, 436 Seventh Avenue, Pittsburgh, PA, 15219-1845, US

(21) Appl. No.: 10/518,215

(22) PCT Filed: Jun. 16, 2003
(86) PCT No.: PCT/KR03/01169

§ 371 Date: Aug. 3, 2005

(30) Foreign Application Priority Data
Jun. 17, 2002 (KR) 10 2002 0033815

(51) Int. Cl. A61L 2/14

(52) U.S. Cl. 422/292; 422/186.21

(57) ABSTRACT

Disclosed is a dehumidifier-equipped plasma sterilization apparatus having a sterilization chamber (10) for receiving therein a sterilization object (11); a high frequency power source (40), connected to a cathode, for generating plasma in optimal condition under control of both an impedance matching controller (42) and an impedance matching circuit (44), the cathode being installed, along with an anode at determined distance, in the sterilization chamber, a vacuum pump (14), connected through an exhaust pipe (116) to the sterilization chamber, for exhausting air from the sterilization chamber (10) to form a vacuum state in the sterilization chamber (10); and a dehumidifier (50), mounted on the exhaust pipe (16) for freeze-condensing the water vapor of the gas flowing through the exhaust pipe. In the apparatus, water vapor remaining in the mixed gas used for sterilizing the sterilization object is frozen and removed so that it does not enter the vacuum pump.

### TECHNICAL FIELD

The present invention relates to a plasma sterilization apparatus for killing any microorganisms present on the surfaces of articles (e.g., medical instruments), which are loaded in a sterilization chamber, using plasma. More particularly, the present invention relates to a dehumidifier-equipped plasma sterilization apparatus in which hydrogen peroxide vapor or a mixture of hydrogen peroxide vapor and air is used as a precursor for germicidally active material and water vapor transformed from the precursor after sterilization is removed by a freezing method in a dehumidifier.

### BACKGROUND ART

There have been developed various apparatuses and methods for sterilizing disposable or recycled medical instruments or tools. Since environment pollution became a prominent figure, extensive and intensive attention has been paid to environment-friendly sterilization apparatuses. One of the most well known environment-friendly sterilization apparatuses is a plasma sterilization apparatus which utilizes a hydrogen peroxide solution as a precursor of germicidally active species because most of its exhausts to the air are hydrogen, oxygen and water vapor.

A prior plasma sterilization apparatus using a hydrogen peroxide solution as a sterilizing agent is introduced in Korean Pat. No. 132233. This plasma sterilization apparatus comprises a sterilization chamber into which gas vaporized from a hydrogen peroxide solution is fed. A vacuum pump is connected to a lower part of the sterilization chamber. In the sterilization chamber, an anode and a cathode are installed, the cathode being connected to a plasma power supply via an impedance matching controller and an impedance matching circuit.

In the plasma sterilization apparatus, the sterilization chamber into which an article to be sterilized (hereinafter referred to as “sterilization object”) is introduced is filled with hydrogen peroxide vapor and then, an electric field is applied by the plasma power supply via the impedance matching controller and the impedance control circuit to generate plasma between the anode and the cathode.

During and after a sterilization process is carried out for a predetermined period of time, the vacuum pump is operated to discharge gas from the sterilization chamber to atmosphere via an exhaust pipe. Most of the discharged gas consists of water vapor, oxygen and hydrogen.

Such a prior plasma sterilization apparatus suffers from a serious disadvantage. Because the vacuum pump is exposed for a long period of time to the oxygen and water vapor contained in abundance in the discharged gas, inner parts of the vacuum pump are corroded by oxidation. In severe cases, the vacuum pump, which is generally expensive, may not operate. Therefore, the vacuum pump needs frequent maintenance and parts change.

### DISCLOSURE OF THE INVENTION

Accordingly, the present invention has been made keeping in mind the above problems occurring in the prior art, and an object of the present invention is to provide a dehumidifier-equipped plasma sterilizing apparatus in which the water vapor contained in the discharged gas after being used for the sterilization of objects is freeze-condensed so as to prevent the entry of the water vapor into the vacuum pump and the corrosion of parts of the vacuum pump, thereby extending the interval between regular maintenance and parts change.

In order to accomplish the above object, the present invention provides a plasma sterilization apparatus, comprising a sterilization chamber for receiving therein a sterilization object, a high frequency power source, connected to a cathode, for generating optimal plasma under control of both an impedance matching controller and an impedance matching circuit, the cathode being installed, along with an anode at a predetermined distance, in the sterilization chamber, and a vacuum pump, connected through a exhaust pipe to the sterilization chamber, for extracting air from the sterilization chamber to form a vacuum state in the sterilization chamber, wherein the exhaust pipe is equipped with a dehumidifier for freeze-condensing the water vapor in the gas flowing through the exhaust pipe to prevent the entry of the water vapor into the vacuum pump.

In accordance with an embodiment, the dehumidifier forms a freezing cycle which comprises a compressor, a condenser, an expansion valve and a freezer, the freezer being housed in a housing connected to the exhaust pipe.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view showing the constitution of a dehumidifier-equipped plasma sterilization apparatus according to the present invention;

FIG. 2 is a partial detail view showing a dehumidifier portion which plays an essential role in the sterilization process of the present invention;

FIG. 3A is a photograph, taken from the plasma sterilization apparatus of the present invention, showing inner parts of the vacuum pump after use for a predetermined period of time; and

FIG. 3B is a photograph, taken from a conventional plasma sterilization apparatus, showing inner parts of the vacuum pump after use for a predetermined period of time.

### BEST MODE FOR CARRYING OUT THE INVENTION

The application of the preferred embodiments of the present invention is best understood with reference to the accompanying drawing.

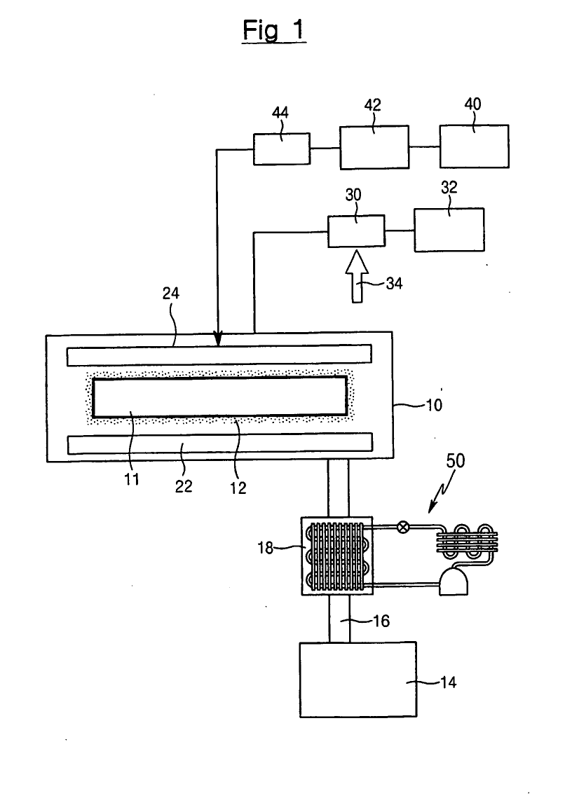

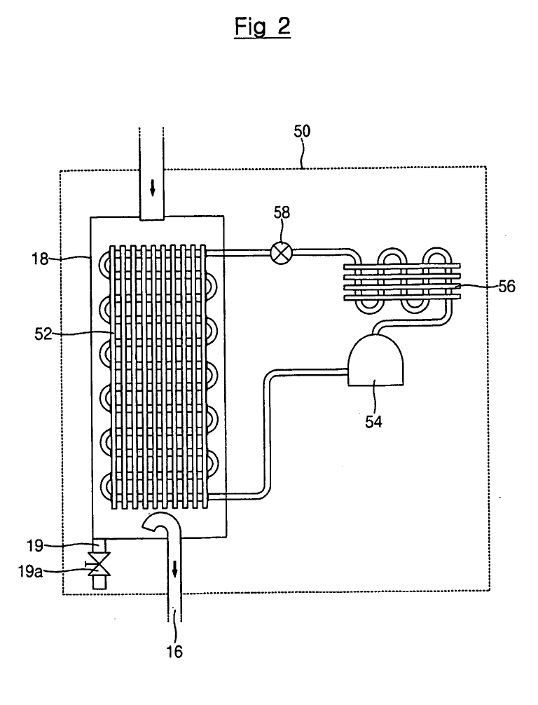

FIG. 1 schematically shows the constitution of a dehumidifier-equipped sterilization apparatus according to the present invention and FIG. 2 is a partial view showing, in detail, the constitution of a dehumidifier, an essential part of the present invention.

As shown in the figures, the sterilization apparatus of the present invention comprises a sterilization chamber 10 for receiving therein a sterilization object 11, such as medical instruments, surgical tools, etc. Before being introduced into the chamber 10, the sterilization object 111 is preferably wrapped with a wrapper 12. Connected via an exhaust line 16 to a lower part of the sterilization chamber 10, a vacuum pump 14 is provided to exhaust air from the sterilization chamber 10 to form a vacuum state.

Inside the sterilization chamber 10 are installed a couple of electrodes. An anode 22 is provided at a lower part of the sterilization chamber 10 while a cathode 24 is at an upper part. The cathode 24 is connected via an impedance matching controller 42 and an impedance matching circuit 44 to a high frequency power source 40 which supplies power with suitable frequencies to generate plasma in optimal conditions.

The frequency of the high frequency power source 40 may fall into a variety of frequency bands. The higher is the frequency, the higher density of the plasma is generated. However, high frequency requires more expensive equipment and additional equipment capable of shielding electromagnetic radiation. Therefore, it is preferable to select a frequency band suitable to equipment in practical use.

To the sterilization chamber 10 is also provided an injection heater 30 which vaporizes a hydrogen peroxide solution 32, heats the vapor along with air 34 and injects the vapor therein.

On the exhaust pipe 16, a dehumidifier 50 is installed which freezes the water vapor of the gas flowing through the exhaust pipe 16, thereby preventing water vapor from getting in the vacuum pump 14. The dehumidifier 50 comprises a housing 18 in which a freezer 52 is installed. The dehumidifier forms a freezing cycle which comprises a compressor 54, a condenser 56, an expansion valve 58 and the freezer 52.

A water drain pipe 19 with an exhaust valve 19a is provided at a lower part of the housing 18. In order to prevent water vapor from getting in the vacuum pump 14, the exhaust pipe 16 is preferably bent into a semicircle or a rise at a lower part of the housing 18 where the exhaust pipe 16 is connected to the housing 18.

A sterilization object 11, e.g., a medical instrument or a surgical tool, is wrapped in the wrapper 12, and loaded into the sterilization chamber 10, and the door of the sterilization chamber 10 is closed. The vacuum pump 14 is operated to exhaust air from the sterilization chamber 10 through the exhaust pipe 16, to the extent that a desired vacuum is formed.

When the vacuum pump 14 creates a desired vacuum inside the sterilization chamber 10, the hydrogen peroxide solution 32 is vaporized and injected, in mixture with air 34, into the sterilization chamber 10 by the injection heater 30.

When the mixture of the hydrogen peroxide vapor 32 and air 34 is filled to a predetermined pressure in the sterilization chamber 10, an electric field is applied through the impedance matching circuit 44 and the impedance controller 42 from the high frequency power source 40 to the cathode 24, thereby generating a high density plasma between the cathode 24 and the anode 22 within the sterilization chamber 10.

By adopting a pulsed power application manner where the high frequency power is applied intermittently, a capacitively-coupled type, high density plasma is generated with a temperature lower than 100° C. Due to such a high frequency power source 40 which is operated in the intermittent application manner, overheating of both the gas within the sterilization chamber 10 and the sterilization object 11 is prevented.

While the vacuum pump 14 is operated continuously, the mixed gas of the hydrogen peroxide solution 32 and the air 34 is injected by the injection heater 30 to maintain the reaction pressure within the sterilization chamber 10.

Reactive species in the high density plasma generated as described above uniformly diffuse throughout the sterilization chamber 10, maintaining a desired plasma atmosphere. The reactive species diffused to the sterilization chamber 10 interact with and thus sterilize the sterilization object 11.

The atmosphere of the inside of the sterilization chamber 10 depends on the high frequency electric power applied to the cathode 24 and the concentration of the mixed gas. Sterilization is finished within the short time period of approximately 5 min from the start of the plasma generation. Although the sterilization process finishes within such a short time, it is preferable to continuously maintain the desired plasma atmosphere for a predetermined time to achieve sufficient sterilization of the sterilization object.

The sterilization efficacy in the sterilization chamber 10 depends on the concentration of the mixed gas produced by the evaporation of the aqueous hydrogen peroxide solution 32 and air 34, i.e., the microbiocidal agents. However, since the sterilization efficiency also depends on the supplied electric power, optimal electric power should be applied to achieve optimal sterilization efficacy.

The wrapper 12 is used to wrap the sterilization object 11 before entry into the sterilization chamber 10. Therefore, the wrapper is selected from materials that are not reactive to the plasma atmosphere and that possesses a fiber-like structure capable of allowing ventilation of plasma there through.

As described above, the sterilization object 11 is completely sterilized by continuously maintaining the plasma atmosphere for a predetermined time.

During the sterilization in the sterilization chamber 10, the dehumidifier 50 is operated in a freeze cycle manner. In detail, as the compressor 54 starts to operate, a refrigerant is highly compressed to a gas of high temperature. The resulting compressed gas refrigerant is transformed into a liquid state of a high pressure by the condenser 56. As passing through the expansion valve 58, the liquid refrigerant is transformed into a vaporized gas. While passing through the freezer 52, the refrigerant absorbs heat from the outside due to its latent heat of vaporization and is returned as a gas of low pressure to the compressor 54. These processes are repeated.

During the freezing cycle, the temperature around the freezer 52 is maintained at −50 to −5° C. as heat is absorbed from the outside by virtue of the latent heat of vaporization. Therefore, when being discharged through the exhaust pipe 16 by the vacuum pump 14, the residual gas passes the housing 18 on the exhaust pipe 16 during which the water vapor of the gas is condensed into liquid water. Therefore, the water vapor is prevented from entering the vacuum pump 14 and thus from eroding parts of the vacuum pump 14 because it is condensed to liquid on the surface of the freezer 52.

After sterilization is complete, the high frequency power source 40 is turned off and the operation of the vacuum pump 14 is ceased to return the sterilization chamber 10 to normal atmospheric pressure. The sterilized wrapped object 11 is removed from the sterilization chamber 10.

As to the freezer 52, its temperature is spontaneously elevated to ambient temperature as the operation of both the vacuum pump 14 and the compressor 54 is stopped. The water condensed in the freezer 52 is thawed and drained through a drain valve 19a of the drainage pipe 19.

### EXPERIMENT

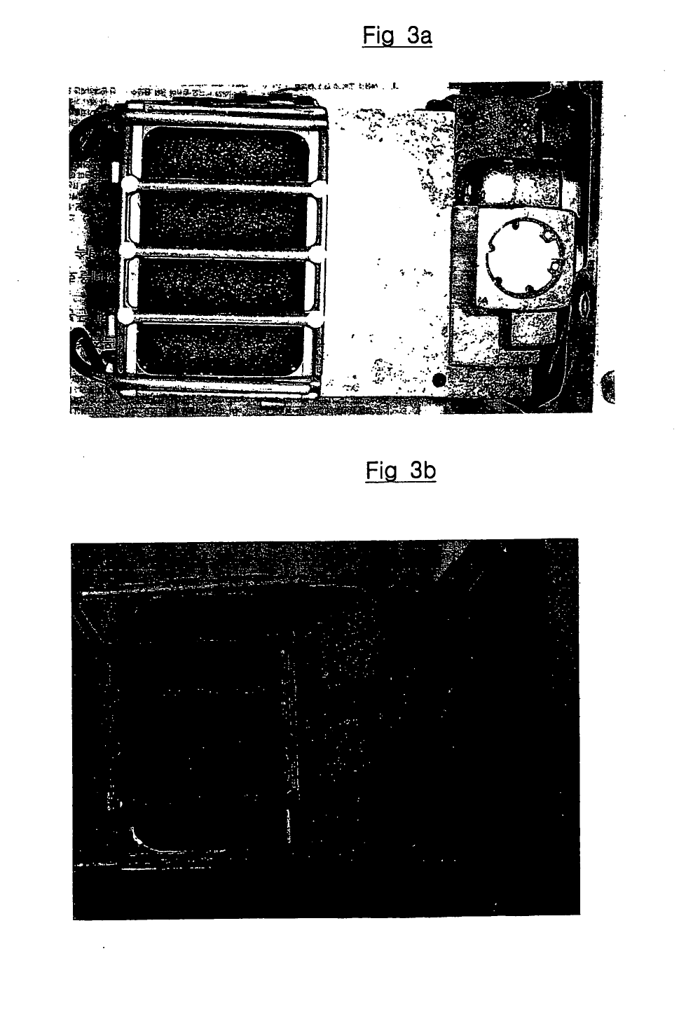

FIGS. 3A and 3B are photographs showing inner parts of vacuum pumps, taken from a dehumidifier-equipped plasma sterilization apparatus of the present invention and a conventional plasma sterilization apparatus equipped with no dehumidifiers, respectively, after sterilization was fulfilled about 450 times (6 times a day for three months).

No traces of corrosion were found in the vacuum pump of the dehumidifier-equipped plasma sterilization apparatus, as seen in the photograph of FIG. 3A, whereas not only the inlet portion but also the whole parts of the vacuum pump of the conventional plasma sterilization apparatus equipped with no dehumidifiers were profoundly corroded, as seen in the photograph of FIG. 3B.

### INDUSTRIAL APPLICABILITY

In the dehumidifier-equipped plasma sterilization apparatus of the present invention, as described above, the water vapor contained in the hydrogen peroxide gas used for the sterilization is condensed by freezing so that it does not enter the vacuum pump. Therefore, the present invention enjoys the advantage of preventing the corrosion of mechanical parts of the vacuum pump, thereby extending the interval between regular maintenance and parts change.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A plasma sterilization apparatus, comprising a sterilization chamber for receiving therein a sterilization object, a high frequency power source, connected to a cathode, for generating optimal plasma under control of both an impedance matching controller and an impedance matching circuit, the cathode being installed, along with an anode at a predetermined distance, in the sterilization chamber, and a vacuum pump, connected through an exhaust pipe to the sterilization chamber, for extracting air from the sterilization chamber to form a vacuum state in the sterilization chamber, wherein the exhaust pipe is equipped with a dehumidifier for freeze-condensing the water vapor in the gas flowing through the exhaust pipe to prevent the entry of the water vapor into the vacuum pump.

2. The plasma sterilization apparatus as set forth in claim 1, wherein said dehumidifier forms a freezing cycle which comprises a compressor, a condenser, an expansion valve and a freezer, the freezer being housed in a housing connected to the exhaust pipe.
